(19) United States
(12) Patent Application Publication
(10) Pub. No.: US 2023/0270766 A1
(43) Pub. Date: Aug. 31, 2023

(54) METHODS AND COMPOSITIONS FOR THE TREATMENT OF ALS

(71) Applicant: CReDO Science, LLC, Austin, TX (US)

(72) Inventor: Ethan Russo, Vashon, WA (US)

(21) Appl. No.: 18/041,015

(22) PCT Filed: Aug. 26, 2021
(86) PCT No.: PCT/US2021/047729

§ 371 Date: Feb. 8, 2023

Related U.S. Application Data
(60) Provisional application No. 63/071,329, filed on Aug. 27, 2020.

(51) Int. Cl. A61K 31/00; A61K 31/015; A61K 31/045; A61P 25/28

(52) U.S. Cl. CPC A61K 31/658; A61K 31/015; A61K 31/045; A61P 25/28

(57) ABSTRACT

The present invention discloses compositions useful for treatment or prevention of Amyotrophic Lateral Sclerosis (ALS) or a related condition, and treatment methods thereof.

### CLAIM OF PRIORITY UNDER 35 U.S.C. § 119

The present Application for Patent claims priority to Provisional Application No. 63/071,329 entitled “METHODS AND COMPOSITIONS FOR THE TREAMENT OF ALS” filed Aug. 27, 2020, and hereby expressly incorporated by reference herein.

### BACKGROUND

### Field

The present invention discloses compositions useful for treatment or prevention of Amyotrophic Lateral Sclerosis (ALS), and treatment methods thereof.

### Background

Neurodegenerative diseases are generally characterized by a degeneration of neurons in either the brain or the nervous system of an individual. ALS, Alzheimer's disease, and multiple sclerosis fall within this category. These diseases are debilitating, the damage that they cause is often irreversible, and the outcome in a number of cases is fatal.

ALS is characterized by gradual degeneration of motor neuron cells in the spinal cord and brain, which ultimately leads to progressive weakness and paralysis of muscle as well as death. ALS occurs in two clinically indistinguishable forms, referred to as a sporadic form and a familial form. The pathogenesis of ALS is not completely understood, although different hypotheses have been suggested, including mitochondria dysfunction, mutation in the superoxide dismutase gene, and defects in neuronal glutamate transport. It has also been hypothesized that autoimmunity is involved in ALS pathogenesis (Appel et al. 1993. J. Neurol. Sci. 118:169-174). In addition, several recent studies have suggested that the immune system may be actively involved in the disease process of ALS, with observations of activated microglia, immunoglobulin G deposits, increased fragment crystallizable receptor expression, and dysregulation of cytokine expression in the spinal cord of ALS patients (Troost et al. 1989. Clin. Neuropathol. 8:289-294; Engelthardt et al. 1990. Arch. Neurol. 47:1210-1216; Schiffer et al. 1996. J. Neurol. Sci. 139(suppl):27-33; Hayashi et al. 2001 J. Neurol. Sci. 188:3-7.9-12).

Recent clinical and pathological studies have shown that involvement outside the motor neuron system is relatively common in ALS (Hayashi et al. 2001, supra; Obal et al. 2001 Neuroreport. 12:2449-2452; Sola et al. 2002 8. Eur. Neurol. 47:108-112; Ono et al. 2001 J. Neurol. Sci. 187:27-34; Alexianu et al. 2001 Neurology. 57:1282-1289.). Additionally, microglia/macrophage activation and inflammatory response have been implicated in ALS disease progression (Appel et al. 1993, supra; Engelthardt et al. 1990, supra; Hayashi et al. 2001 supra; Obal et al. 2001 Neuroreport. 12:2449-2452; McGeer et al. 2002 Muscle Nerve. 26:459-47028, 29). However, despite intensive investigation, ALS has no known cause or effective therapy.

### SUMMARY

Some embodiments of the invention relate to a composition that can include a combination of at least two active agents extracted from Cannabis and a pharmaceutically acceptable carrier. The composition can be provided in a form suitable for transmucosal administration. The two active agents can have a synergistic effect on the treatment of ALS or a related condition and/or symptoms thereof.

In some embodiments, at least one of the active agents is a cannabinoid, a terpene, or a flavonoid.

In some embodiments, the active agents can be selected from THC, CBD, CBG, THCA, CBDA, beta-caryophyllene, alpha-pinene, D-limonene, and linalool.

In some embodiments, the composition can include THC, CBD, CBG, THCA, CBDA, beta-caryophyllene, alpha-pinene, D-limonene, and linalool.

In some embodiments, the active agents can include THC and CBD.

In some embodiments, the active agents can include THC and beta-caryophyllene.

In some embodiments, the active agents can include CBD and beta-caryophyllene.

In some embodiments, the active agents can include CBD and linalool.

In some embodiments, the active agents can include THC and alpha-pinene.

In some embodiments, the composition can include cannflavin A.

In some embodiments, the pharmaceutically acceptable carrier can include aqueous solution, non-aqueous solution, or a combination of an aqueous solution and a non-aqueous solution thereof.

In some embodiments, the pharmaceutically acceptable carrier can include a solution, gel, suspension, liposomal dispersion, emulsion, microemulsion, nanoparticle, or combinations thereof.

In some embodiments, the composition can be provided in a sublingual or buccal or oromucosal transmucosal liquid dosage form. In some embodiments, the transmucosal dosage form can be a spray or liquid drops.

In some embodiments, the composition can be provided in a sublingual or buccal or oromucosal transmucosal solid dosage form. In some embodiments, the transmucosal dosage form can be a tablet, a chewing gum, a patch, a lozenge, a troche, a pastille, a sachet, and a rapid disintegrating tablet, strip, and/or the like.

In some embodiments, the composition can further include at least one of: flavor agent, artificial coloring, sweetener, lubricating agent, disintegration agent, permeation enhancer, lubricating agent, diluent, base, buffering agent or the like.

In some embodiments, the composition can further include an additional active agent.

Some embodiments of the invention relate to a method for the treatment of ALS or a related condition and/or symptoms of ALS or a related condition. Th method can include transmucosally administrating a therapeutically effective amount of the composition to a subject in need of such treatment. In some embodiments, ALS or a related condition and/or symptoms of ALS or a related condition can ameliorated after treatment.

In some embodiments, the pharmaceutical composition can be administered transmucosally through sublingual, buccal, or oromucosal routes of delivery.

### BRIEF DESCRIPTION OF THE DRAWINGS

FIG. 1 is a table of pairwise combinations of synergistic ingredients of the invention wherein the ingredients are cannabinoids, flavonoids, or a cannabinoid and a flavonoid.

FIG. 2 is a table of pairwise combinations of synergistic ingredients of the invention wherein the ingredients are terpenes.

FIG. 3 is a table of pairwise combinations of synergistic ingredients of the invention wherein the ingredients are a terpene and a cannabinoid or a terpene and a flavonoid.

FIG. 4 is a table listing major ratios of pairwise combinations of synergistic ingredients of the invention, named as ratios A through S.

FIG. 5 is a table listing additional synergistic ingredients that are added to the compositions in certain embodiments.

### DETAILED DESCRIPTION

The present invention relates to compositions useful for treatment or prevention of ALS or related medical disorders or conditions and/or symptoms of ALS or related medical disorders or conditions, and treatment methods thereof.

The term “ALS related disorder” or “ALS related condition” can refer to any motor neuron disorder such as Primary Lateral Sclerosis (PLS), Progressive Muscular Atrophy (PMA), Progressive Bulbar Palsy (PBP), as well as Fronto Temporal Dementia (FTD), and/or the like.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All references, publications, patents, patent applications, and commercial materials mentioned herein are incorporated herein by reference for all purposes, including for describing and disclosing the cell lines, vectors, and methodologies which are reported in the publications which might be used in connection with the invention. Nothing herein is to be construed as an admission that the invention is not entitled to antedate such disclosure.

In order to provide a clear and consistent understanding of the specification and claims, including the scope to be given such terms, the following definitions are provided:

The term “administration” of the pharmaceutically active agents or active compounds and the pharmaceutical compositions defined herein includes but is not limited to transmucosal application. As used herein, the terms “active agents” and “active compounds” can be used interchangeably. Sublingual, buccal, oral, and oromucusal administration are particularly preferred in the present invention.

“Ameliorate” and/or “amelioration” means a lessening of the detrimental effect or severity of the disease in the subject receiving therapy. The severity of the response can be determined by means that are well known in the art.

The terms “effective amount” and/or “pharmaceutically effective amount” refer to a nontoxic but sufficient amount of the agent to provide the desired biological result. That result can be reduction and/or alleviation of the signs, symptoms, or causes of a disease, such as neural diseases and malignant hyperthermia, or any other desired alteration of a biological system. Such amounts are described below. An appropriate “effective” amount in any individual case may be determined by one of ordinary skill in the art using routine experimentation.

As used herein, the term “excipient” means the substances used to formulate active pharmaceutical ingredients (API) into pharmaceutical formulations; in a preferred embodiment, an excipient does not lower or interfere with the primary therapeutic effect of the API. Preferably, an excipient is therapeutically inert. The term “excipient” can include, but need not be limited to, carriers, diluents, vehicles, solubilizers, stabilizers, bulking agents, acidic or basic pH-adjusting agents, and binders. Excipients can also be those substances present in a pharmaceutical formulation as an indirect or unintended result of the manufacturing process. Preferably, excipients are approved for or considered to be safe for human and animal administration, i.e., GRAS substances (generally regarded as safe). GRAS substances are listed by the Food and Drug Administration in the Code of Federal Regulations (CFR) at 21 CFR 182 and 21 CFR 184, incorporated herein by reference.

As used herein, the term “formulate” refers to the preparation of a drug in a form suitable for administration to a mammalian patient, e.g., a human. Thus, “formulation” can include the addition of one or more pharmaceutically acceptable excipients, diluents, carriers, and/or pH-adjusting agents.

The terms “pharmaceutically acceptable” and/or “pharmacologically acceptable” means a material which is not biologically or otherwise undesirable, i.e., the material can be administered to an individual without causing any undesirable biological effects or interacting in a deleterious manner with any of the components of the composition in which it is contained.

As used herein, a “pharmaceutically acceptable carrier” is a material that is nontoxic, generally inert, and does not affect the functionality of the active ingredients adversely. Examples of pharmaceutically acceptable carriers are well known, and they are sometimes referred to as diluents, vehicles, or excipients. The carriers can be organic or inorganic in nature. In addition, the formulation can contain additives such as flavoring agents, coloring agents, thickening or gelling agents, emulsifiers, wetting agents, buffers, stabilizers, and preservatives such as antioxidants.

The term “pharmaceutical composition” as used herein means a composition that is made under conditions such that it is suitable for administration to humans, e.g., it is made under GMP conditions and contains pharmaceutically acceptable excipients, including, without limitation, stabilizers, pH-adjusting agents, bulking agents, buffers, carriers, diluents, vehicles, solubilizers, and binders.

“Substantially” can be defined as being largely but not necessarily wholly what is specified, as understood by a person of ordinary skill in the art.

The liquid carrier and/or vehicle can be a solvent or liquid dispersion medium comprising, for example, water; ethanol; a polyol such as glycerol, propylene glycol, or liquid polyethylene glycols, and the like; vegetable oils; nontoxic glyceryl esters; and suitable mixtures thereof. The prevention of the growth of microorganisms can be accomplished by various antibacterial and antifungal agents, for example, parabens, chlorobutanol, phenol, sorbic acid, thimerosal, and the like.

As used herein, the terms “treating” and/or “treatment” of a disease include preventing the disease, i.e. preventing clinical symptoms of the disease in a subject that can be exposed, or predisposed, to the disease, but does not yet experience or display symptoms of the disease; and inhibiting the disease, i.e., arresting the development of the disease or its clinical symptoms, such as by suppressing or relieving the disease, i.e., causing regression of the disease or its clinical symptoms.

Within the context of this invention, the term “treatment” of a disorder can include the therapy, prevention, prophylaxis, retardation or reduction of pain provoked by the disorder. The term treatment can include, in particular, the control of disease progression and associated symptoms. In relation to ALS, the term treatment can also designate a retardation or delayed onset of paralysis, a reduction or prevention of motor neuron degeneration, a reduction of pain, and/or an increase in survival.

### General

The present invention provides compositions and methods for the delivery of a composition including active agents to a patient in need of such treatment, comprising the transmucosal administration of active agents. More specifically, the invention features dosage forms, compositions, and methods for the transmucosal delivery of active agents through sublingual, buccal, oral, and/or oromucosal routes of administration for the treatment of ALS or a related condition and/or symptoms of ALS or a related condition. The dosage forms are those suitable for transmucosal drug delivery, such as spray, drops, gels, tablets, troches, lozenges, chewing gum, patches, etc.

The invention is directed to a pharmaceutical composition that can include about 0.05 mg to about 200 mg of active agents. For example, the composition can include about 0.05, 1, 10, 25, 50, 100, 125, 150, 175, or 200 mg of active agents. In some embodiments, the pharmaceutical composition comprises from about 2 mg to about 100 mg of active agents. For example, the composition can include about 2, 5, 10, 20, 30, 40, 50, 60, 70, 80, 90, or 100 mg of active agents.

In some embodiments, the present invention relates to a composition for pharmaceutical drug delivery. The composition can be formulated to be suitable for systemic application, for example, transmucosal application. The composition can comprise a therapeutically-effective amount of active agents.

In some embodiments, the composition comprises one or more gelling or thickening agents. Exemplary gelling agents include, but are not limited to, carbomer; carboxyethylene or polyacrylic acid such as carbomer 980 and/or 940 NF, 981 and/or 941 NF, 1382 and/or 1342 NF, 5984 and/or 934 NF, ETD 2020, 2050, 934P NF, 971P NF, 974P NF; polycarbophils such as NOVEON AA-1 (polycarbophil USP), NOVEON CA1/CA2 (calcium polycarbophil); carbomer copolymers such as PEMULEN TR1 NF (carboxypolymethylene) and/or PEMULEN TR2 NF (carboxypolymethylene); carbomer interpolymers such as CARBOPOL ETD 2020 NF, CARBOPOL ETD 2050 NF, CARBOPOL ULTRA EZ 10, etc.; cellulose derivatives such as ethylcellulose, hydroxypropylmethylcellulose (HPMC), ethyl-hydroxyethylcellulose (EHEC), carboxymethylcellulose (CMC), hydroxypropylcellulose (HPC), hydroxyethylcellulose (HEC), etc.; natural gums such as arabic, xanthan, guar gums, alginates, etc.; polyvinylpyrrolidone derivatives; polyoxyethylene polyoxypropylene copolymers, etc.; others like chitosan, polyvinyl alcohols, pectins, veegum grades, and the like. Other suitable gelling agents to apply to the present invention can include, but are not limited to, carbomers. Alternatively, other gelling or thickening agents known by those skilled in the art can also be used. The gelling agent or thickener can be present from about 0.2 to about 30% w/w depending on the type of polymer, as known by one skilled in the art. For example, the gelling agent or thickener can be present in about 0.2, 0.5, 1, 3, 5, 7, 9, 10, 15, 20, 25, or 30% w/w. A concentration range of the gelling agent(s), for example, hydroxypropyl cellulose or carbomer, can be a concentration of between about 0.5 and about 5.0 weight percent (e.g., 0.5, 0.75, 1.0, 1.5, 2.0, 2.5, 3.0, 3.5, 4.0, 4.5, or 5.0 weight percent), or between about 1.0 and about 3.0 weight percent (e.g., 1.0, 1.25, 1.5, 1.75, 2.0, 2.25, 2.5, 2.75, or 3.0 weight percent).

In some embodiments, the composition can include a permeation enhancer, also known in the art as a penetration enhancer. Permeation enhancers can include, but are not limited to, sulfoxides such as dimethylsulfoxide and decylmethylsulfoxide; surfactants such as sodium laurate, sodium lauryl sulfate, cetyltrimethylammonium bromide, benzalkonium chloride, poloxamer (231, 182, 184), tween (20, 40, 60, 80) (polysorbate), lecithin, and/or the like; 1-substituted azacycloheptan-2-ones, particularly 1-n-dodecylcyclazacycloheptan-2-one; fatty alcohols such as lauryl alcohol, myristyl alcohol, oleyl alcohol, and the like; fatty acids such as lauric acid, oleic acid, valeric acid, and the like; fatty acid esters such as isopropyl myristate, isopropyl palmitate, methylpropionate, ethyl oleate, and the like; polyols and esters thereof such as propylene glycol, ethylene glycol, glycerol, butanediol, polyethylene glycol, and polyethylene glycol monolaurate; amides and other nitrogenous compounds such as urea, dimethylacetamide (DMA), dimethylformamide (DMF), 2-pyrrolidone, 1-methyl-2-pyrrolidone, ethanolamine, diethanolamine, triethanolamine, and terpenes; alkanones and organic acids, particularly salicylic acid and salicylates, citric acid, and succinic acid. As noted earlier herein, Percutaneous Penetration Enhancers, Smith et al. (Eds.). (CRC Press, 1995), which is incorporated herein by reference thereto, provides an excellent overview of the field and further information concerning possible secondary enhancers for use in conjunction with the present invention. More permeation enhancer(s) suitable to be used with the present invention can be known by those skilled in the art. The permeation enhancer can be present from about 0.1 to about 30% w/w depending on the type of compound. For example, the permeation enhancer can be present in about 0.1, 1, 3, 5, 7, 9, 10, 15, 20, 25, or 30% w/w.

In some embodiments, the permeation enhancer can include, but need not be limited to, a bile salt, sodium dodecyl sulfate, dimethyl sulfoxide, sodium lauryl sulfate, a derivative of a saturated or unsaturated fatty acid, a surfactant, a bile salt analog, and/or a derivative of a bile salt. In some embodiments, the permeation enhancer can be a synthetic permeation enhancer.

In another embodiment, the composition can include antioxidant(s), for example, tocopherol and derivatives, ascorbic acid and derivatives, butylated hydroxyanisole, butylated hydroxytoluene, fumaric acid, malic acid, propyl gallate, sodium metabisulfite, and derivatives, and combinations thereof, in a concentration of about 0.01 to about 5 weight percent (e.g., 0.01, 0.1, 1.0, 1.5, 2.0, 2.5, 3.0, 3.5, 4.0, 4.5, or 5.0 weight percent) or a concentration of about 0.1 to about 0.5 weight percent (e.g., 0.1, 0.15, 0.2, 0.25, 0.3, 0.35, 0.4, 0.45, or 0.5 weight percent), depending on the type of antioxidant used, as known by the one skilled in the art.

In another embodiment, the composition can include buffering agent(s), for example, carbonate buffers, citrate buffers, phosphate buffers, acetate buffers, hydrochloric acid, lactic acid, tartaric acid, inorganic and organic bases, and combinations thereof in a concentration of about 1 to about 10 weight percent (e.g., 1.0, 2.0, 3.0, 4.0, 5.0, 6.0, 7.0, 8.0, 9.0, or 10 weight percent) or about 2 to about 5 weight percent (e.g., 2.0, 2.5, 3.0, 3.5, 4.0, 4.5, or 5.0 weight percent), depending on the type of buffering agent(s) used, as known by the one skilled in the art. The concentration range of said buffering agents can be those enabling design of compositions having a pH close to the physiologic pH of the mucosal membranes, between about pH 2.0 and about pH 10.0 (e.g., 2.0, 3.0, 4.0, 5.0, 6.0, 7.0, 8.0, 9.0, or 10 pH) or between about pH 3.0 and about pH 7.0 (e.g., 3.0, 3.5, 4.0, 4.5, 5.0, 5.5, 6.0, 6.5, or 7.0 pH). Concentrations of the buffering agent(s) can vary, however, as known by the one skilled in the art. The buffering agent can replace up to 100% of the water amount within the composition.

The transmucosal pharmaceutical formulation of the present invention can also further include, but need not be limited to, preservatives such as benzalkonium chloride and derivatives, benzoic acid, benzyl alcohol and derivatives, bronopol, parabens, centrimide, chlorhexidine, cresol and derivatives, imidurea, phenol, phenoxyethanol, phenylethyl alcohol, phenylmercuric salts, thimerosal, sorbic acid and derivatives, and combinations thereof. The preservative is present from about 0.01 to about 10% w/w depending on the type of compound used, as known by the one skilled in the art. For example, the preservative can be present in about 0.01, 0.5, 1.0, 2.0, 3.0, 4.0, 5.0, 6.0, 7.0, 9.0, 9.0, 10% w/w.

The transmucosal pharmaceutical formulation of the present invention can also further include, for example, humectants, sequestering agents, moisturizers, surfactants, emollients, colorants, fragrances, flavors, or any combination thereof.

In some embodiments, the transmucosal dosage form is a liquid formulation comprising active agents, aqueous solvent, and a polar organic solvent, wherein the polar organic solvent is present in an amount sufficient to enhance the solubility of the active agents.

In some embodiments, a gel formulation of the present invention can comprise a therapeutically effective amount of active agents, of between about 0.01 to about 5 weight percent. For example, the active agents can be present in about 0.01, 0.1, 1.0, 1.5, 2.0, 2.5, 3.0, 3.5, 4.0, 4.5, or 5.0 weight percent. The primary vehicle can include between about 10 to about 60 weight percent of water (e.g., 10, 15, 20, 25, 30, 35, 40, 45, 50, 55, or 60 weight percent of water), between about 30 to about 70 weight percent ethanol (e.g., 30, 35, 40, 45, 50, 55, 60, 65, or 70 weight percent of ethanol), between about 15 and about 60 weight percent of a 10:1 to 1:10 (weight to weight) mixture of diethylene glycol mono ethyl ether and propylene glycol (e.g., 15, 20, 25, 30, 35, 40, 45, 50, 55, or 60 weight percent), and between about 0.1 and about 2 weight percent of lauryl alcohol, myristyl alcohol, oleyl alcohol, lauric acid, myristic acid, or oleic acid (e.g., 0.1, 0.25, 0.5, 0.75, 1.0, 1.25, 1.5, 1.75, 2.0 weight percent). The primary vehicle can be gellified with between about 0.5 and about 5 weight percent of hydroxypropylcellulose. For example, the hydroxypropylcellulose can be present in about 0.5, 1.0, 1.5, 2.0, 2.5, 3.0, 3.5, 4.0, 4.5, or 5.0 weight percent. The apparent pH of the gel can be between about pH 2.0 and about pH 10.0 (e.g., 2.0, 3.0, 4.0, 5.0, 6.0, 7.0, 8.0, 9.0, or 10 pH) or between about pH 3.0 and about pH 7.0 (e.g., 3.0, 3.5, 4.0, 4.5, 5.0, 5.5, 6.0, 6.5 or 7.0 pH).

In another embodiment, the active agents can be present at a dose of 0.05-5 mg of active agents. For example, the active agents can be present in about 0.05, 0.1, 0.5, 1.0, 1.5, 2.0, 2.5, 3.0, 3.5, 4.0, 4.5, or 5.0 mg. The alcohol can include, but is not limited to, ethanol, propylene glycol, glycerol, polyethylene glycol, and mixtures thereof. The polar organic solvent can be present in an amount of 0.5-50% w/w. For example, the polar organic solvent can be present in about 0.5%, 1%, 5%, 10%, 15%, 20%, 25%, 30%, 35%, 40%, 45%, or 50% w/w.

In addition, the transmucosal delivery system of the pharmaceutical composition can include a buffer to maintain the pH of the formulation and a pharmaceutically acceptable thickening agent. The pharmaceutical composition can further include one or more pharmaceutical excipients and can further include a pharmaceutically acceptable preservative.

The buffer of the transmucosal delivery system can include, but is not limited to, acetate, citrate, prolamine, carbonate, phosphate buffers, and combinations thereof.

The thickening agent of the transmucosal delivery system can include but need not be limited to, one or more methyl cellulose, xanthan gum, carboxymethyl cellulose, hydroxypropyl cellulose, carbomer, polyvinyl alcohol, alginates, acacia, chitosans, and combinations thereof.

The formulation can further comprise a sweetener suitable for sublingual and buccal delivery systems. The sweetener can be, but is not limited to, mannitol, saccharin, saccharin sodium, and combinations thereof. The formulation can further comprise a flavoring agent. For example, the flavoring agent can be menthol or the like. The formulation can further comprise a thickening agent. The thickening agent can be, but is not limited to, one or more methyl cellulose, xanthan gum, carboxymethyl cellulose, hydroxypropyl cellulose, carbomer, polyvinyl alcohol, alginates, acacia, chitosans, and combinations thereof.

In some embodiments, the transmucosal carrier of the transmucosal dosage unit can be an aqueous solution. Further, the aqueous solution can be, but is not limited to, one or more aqueous gels, aqueous suspensions, aqueous liposomal dispersions, aqueous emulsions, aqueous microemulsions, aqueous nanoparticles, and combinations thereof.

Alternatively, the carrier of the transmucosal dosage unit can be a non-aqueous solution. The non-aqueous solution can be selected from a group including, but not limited to, one or more non-aqueous gels, non-aqueous suspensions, non-aqueous liposomal dispersions, non-aqueous emulsions, non-aqueous microemulsions, non-aqueous nanoparticles, and combinations thereof.

The carrier of the transmucosal dosage unit can also be a combination of an aqueous solution and a non-aqueous solution. The formulation can be partially pressurized. Alternatively, the carrier of the transmucosal dosage unit can be a powder formulation.

The powder formulation can be selected from, but need not be limited to, one or more simple powder mixtures, powder microspheres, coated powder microspheres, liposomal dispersions, and combinations thereof.

In some embodiments the oral transmucosal dosage form can be chosen from: a chewing gum, a patch, a lozenge, a tablet, a troche, a pastille, a sachet, a rapid disintegrating tablet, and the like.

The formulations of the present invention can be provided in a unit dose container(s). Such containers can include inner and outer surfaces, wherein the formulation of the present invention is contained by the inner surface of the container. In selected embodiments, the container can be a packet or a vial, and the inner surface of the container can further comprise a liner. For example, in some embodiments, the container is a flexible, foil packet and the liner is a polyethylene liner. Alternatively, or in addition, the formulations of the present invention can be provided in a multiple dose container(s). Such multiple dose containers can include inner and outer surfaces, wherein the gel for pharmaceutical drug delivery is contained by the inner surface of the container. Multiple dose containers can, for example, dispense fixed or variable metered doses. Multiple dose containers can, for example, be a stored-energy metered dose pump or a manual metered dose pump.

In another aspect, the present invention can include a composition for pharmaceutical drug delivery, comprising a therapeutically effective amount of active agents in a hydroalcoholic vehicle comprising water, a short chain alcohol, a monoalkyl ether of diethylene glycol, a pharmaceutically acceptable glycol, and an optional fatty permeation enhancer. In such compositions the pH of the composition can be between about pH 2.0 and about pH 9.0 (e.g., 2.0, 2.5, 3.0, 3.5, 4.0, 4.5 5.0, 5.5, 6.0, 6.5, 7.0, 7.5, 8.0, 8.5, or 9.0 pH). These compositions for pharmaceutical delivery can include further components as described herein, for example, the hydroalcoholic vehicle can further comprise a permeation enhancer. Such compositions can be formulated in a variety of ways including wherein the hydroalcoholic vehicle is gellified. These compositions can be used, for example, for transmucosal applications including application to sublingual and buccal tissues.

In yet another aspect, the present invention can comprise a composition for pharmaceutical drug delivery, comprising a therapeutically effective amount of active agents in a hydroalcoholic vehicle comprising water, a short chain alcohol, a monoalkyl ether of diethylene glycol, a pharmaceutically acceptable glycol, and an optional fatty permeation enhancer. Compositions for pharmaceutical delivery can include further components as described herein. For example, the hydroalcoholic vehicle can further comprise a cosolvent(s), a penetration enhancer(s), a buffering agent(s), a preservative(s), an emollient(s), a humectant(s), and/or a gelling agent(s). Such compositions can be formulated in a variety of ways including wherein the hydroalcoholic vehicle is gellified. These compositions can be used, for example, for transmucosal applications including application to sublingual and buccal tissues.

In a further aspect, the present invention can include methods of manufacturing the compositions described herein for pharmaceutical drug delivery. In some embodiments, the method of manufacturing can comprise mixing the components to yield a substantially homogeneous gel, wherein the pH of the gel is between about pH 4.5 and about pH 8.5 (e.g., 4.5 5.0, 5.5, 6.0, 6.5, 7.0, 7.5, 8.0, or 8.5 pH). Exemplary components can include, but are not limited to, the following: a therapeutically effective amount of active agents, a primary vehicle comprising water, at least one short-chain alcohol, a monoalkyl ether of diethylene glycol, a pharmaceutically acceptable glycol, an optional fatty permeation enhancer. These methods can include addition of further components as described herein, for example, the hydroalcoholic vehicle can further comprise cosolvent(s), penetration enhancer(s), buffering agent(s), preservative(s), emollient(s), humectant(s), and/or gelling agent(s). The method can provide a gel suitable for transmucosal delivery of active agents. Further, a method of manufacturing can further include dispensing the pharmaceutical composition into one or more containers (for example, a unit dose container [e.g., a flexible, foil packet, further comprising a liner] or a multiple dose container).

In another aspect, the present invention can include methods for administering active agents to a human or animal subject in need thereof. For example, the method can comprise providing a composition of the present invention for transmucosal, pharmaceutical delivery of active agents. Doses of the compositions of the present invention can, for example, be a gel applied to the sublingual, buccal, oromucosal, and/or oral tissues. Further, doses of the compositions of the present invention can be applied in single or divided doses. In some embodiments, the composition can be applied as one or more daily doses of the gel or spray to sublingual, buccal, oromucosal, and/or oralmucosa of the subject in an amount sufficient for the active agents to achieve therapeutic concentration in the bloodstream. Further dosage forms of the compositions of the present invention can be determined by one of ordinary skill in the art in view of the teachings presented herein. The compositions of the present invention can be applied to a mucosal membrane using a variety of means, including, but not limited to a pump-pack, a brush, a swab, a finger, a hand, and/or another applicator.

The methods of manufacturing of the present invention can include dispensing compositions of the present invention into appropriate containers. The compositions of the present invention can be packaged, for example, in unit dose or multi-dose containers. The container can define an inner surface that contains the composition. Any suitable container can be used. The inner surface of the container can further comprise a liner or be treated to protect the container surface and/or to protect the composition from adverse effects that can arise from the composition being in contact with the inner surface of the container. Exemplary liners or coating materials can include, but are not limited to, high density polyethylene, low density polyethylene, very low density polyethylene, polyethylene copolymers, thermoplastic elastomers, silicon elastomers, polyurethane, polypropylene, polyethylene terephthalate, nylon, flexible polyvinylchloride, natural rubber, synthetic rubber, and combinations thereof. Liners or coating material can be substantially impermeable to the composition and to the individual components of the composition.

A number of types of containers are known in the art, such as, for example, packets with rupturable barriers (see, for example, U.S. Pat. Nos. 3,913,789; 4,759,472; 4,872,556; 4,890,744; 5,131,760; and 6,379,069), single-use packets (see, for example, U.S. Pat. Nos. 6,228,375 and 6,360,916), tortuous path seals (see, for example, U.S. Pat. Nos. 2,707,581; 4,491,245; 5,018,646; and 5,839,609), and various sealing valves (see, for example, U.S. Pat. Nos. 3,184,121; 3,278,085; 3,635,376; 4,328,912; 5,529,224; and 6,244,468). The entire contents of the foregoing are fully incorporated by reference herein. One example of a unit dose container is a flexible foil packet with a polyethylene liner.

Containers/delivery systems for the compositions of the present invention can also include a multi-dose container providing, for example, a fixed or variable metered dose application. Multi-dose containers can include, but are not limited to, a metered dose aerosol, a stored-energy metered dose pump, or a manual metered dose pump. In some embodiments, the container/delivery system can be used to deliver metered doses of the compositions of the present invention for application to the sublingual and buccal mucosa of a subject. Metered dose containers can comprise, for example, an actuator nozzle that accurately controls the amount and/or uniformity of the dose applied. The delivery system can be propelled by, for example, a pump pack or use of propellant(s) (e.g., hydrocarbons, hydro fluorocarbons, nitrogen, nitrous oxide, and/or carbon dioxide). Propellants can include but need not be limited to those of the hydrofluorocarbon (e.g., hydrofluoroalkanes) family, which are considered more environmentally friendly than the chlorofluorocarbons. Exemplary hydrofluoroalkanes include, but are not limited to, 1,1,1,2-tetrafluoroethane (HFC-134(a)), 1,1,1,2,3,3,3-heptafluoropropane (HFC-227), difluoromethane (HFC-32), 1,1,1-trifluoroethane (HFC-143(a)), 1,1,2,2-tetrafluoroethane (HFC-134), 1,1-difluoroethane (HFC-152a), as well as combinations thereof. Many pharmaceutically acceptable propellants have been previously described and can be used in the practice of the present invention in view of the teachings presented herein. The delivery system can provide dose uniformity. In some embodiments, airless packaging with excellent barrier properties can be used to prevent degradation of active agents, for example, airless metered-dose pumps wherein the composition comprising active agents is packaged in collapsible aluminum foils. Accurate dosing from such pumps ensures reproducibility of dose.

The present invention can further include methods for administering a composition of the present invention to a subject in need thereof. Compositions of the present invention comprising active agents can be employed, for example, for the treatment of a variety of conditions and/or disease states which have been historically treated by oral doses of active agents.

Transmucosal delivery generally has better patient compliance, less risk of infection, and lower cost than invasive procedures such as injection and implantation. It also has much shorter onset time, i.e., the time from administration to therapeutic effect, than oral delivery. A drug absorbed via the buccal and sublingual mucosa will also avoid first pass metabolism, in which the drug is metabolized in the gastrointestinal tract and liver. Similarly, a drug absorbed via these routes avoids the variability in gastric emptying time commonly observed in patients with proximal gastrointestinal motility syndromes, allowing for greater predictability in obtaining therapeutic blood levels. Such transmucosal delivery systems are simple and dosage forms can be administered by the caregiver or patient with minimal discomfort.

The compositions according to the present invention can be administered, for example, as a spray, drop, suspension, gel, ointment, cream, and/or powder.

The administration of a composition can also include using a pressurized container, strip, dropper, and the like containing a composition of the present invention.

The compositions according to the present invention can be administered, for example, as solid sublingual and/or buccal transmucosal delivery system such as, but not limited to, a chewing gum, a patch, a lozenge, a tablet, a troche, a pastille, a sachet, and/or a rapid disintegrating tablet or strip.

In the present compositions, many other excipients known in the art can be added, such as one or more preservatives, surfactants, co-solvents, adhesives, antioxidants, buffers, viscosity enhancing agents, and agents to adjust the pH and the osmolarity.

As set forth previously, the solid transmucosal delivery systems that can be used with the present invention can take forms including, but not limited to, a powder, a chewing gum, a patch, a lozenge, a tablet, a troche, a pastille, a sachet, and a rapid disintegrating tablet or strip.

In some embodiments, the formulation can be presented, for example, as a capsule, tablet, caplet, pill, powder, granule, or a suspension suitable for transmucosal delivery prepared by conventional means with one or more pharmaceutically acceptable excipients, e.g., with conventional additives or fillers such as lactose, mannitol, corn starch, potato starch, and/or combinations thereof; with binders or binding agents such as crystalline cellulose, cellulose derivatives, acacia, corn starch (including pregelatinized), gelatins, and/or combinations thereof; with disintegrators or disintegrants such as corn starch, potato starch, sodium carboxymethyl-cellulose, and/or combinations thereof; or with lubricants and/or wetting agents such as talc and/or magnesium stearate. Tablets can be coated by methods well known in the art. The formulation can be presented as an immediate-release, slow-release, sustained-release, or controlled-release form. In another embodiment, the formulation can be presented as liquid preparations for oral administration and can take the form of, for example, solutions, syrups, or suspensions, or they can be presented as a dry product for constitution with water or another suitable vehicle before use. Such liquid preparations can be prepared by conventional means with pharmaceutically acceptable additives such as suspending agents (e.g., sorbitol syrup, methyl cellulose, or hydrogenated edible fats); emulsifying agents (e.g., lecithin or acacia); and/or non-aqueous vehicles (e.g., methyl, propyl-p-hydroxybenzoates, or sorbic acid).

For transmucosal administration, the active agents can be combined with penetration enhancers such as propylene glycol, polyethylene glycol, nerolidol, ethanol, oleic acid, N-methylpyrrolidone, or the like, and/or combinations thereof, which can increase the permeability of the mucosa to the active agents and permits the active agents to penetrate through the mucosal membranes and into the bloodstream. The compound/enhancer compositions can also be combined with a polymeric substance such as ethylcellulose, hydroxypropyl cellulose, ethylene/vinylacetate, polyvinyl pyrrolidone, or the like and/or combinations thereof, to provide the composition in gel form, which can be dissolved in solvent such as methylene chloride, evaporated to the desired viscosity, and then applied to sublingual, and/or buccal mucosa.

The composition can further include additional pharmaceutical ingredients to provide desirable characteristics, such as aesthetically pleasing qualities, improved taste, and the like, to otherwise render the dosage formulation more likely to be administered by the patient. Examples of desirable ingredients can include, without limitation, one or more penetration enhancers, colorants, flavorings agents, solvents and co-solvents, coating agents, direct compression excipients, disintegrants, glidants, lubricants, polishing agents, suspending agents, sweetening agents, anti-adherents, binders, and diluents. The ingredients can also include one or more preservatives, emulsifying agents, antioxidants, plasticizers, surfactants, tonicity agents, viscosity increasing agents, and combinations thereof. Examples of useful additives include, without limitation, propylene glycol; polyethylene glycol; flavors such as, for example, orange, cherry, mint, strawberry, and the like; and other commonly utilized ingredients.

The components of the composition can be formulated in any suitable orally dissolvable dosage form to deliver the active agents to the sublingual and/or buccal oral mucosal tissue. For example, suitable formulations can include, without limitation, solid formulations such as a lozenge, a lollipop, a troche, a chewable gum, a solid candy, a granular solid, a chewable tablet or pill, an orally disintegrated tablet, an orally disintegrated pill, an orally disintegrated strip, an orally dissolvable tablet, an orally dissolvable strip, and an orally dissolvable pill.

Such formulations can be prepared utilizing formulating procedures known in the art. For example, there are several ways to create a solid, orally dissolvable formulation, including but not limited to, wet granulation, co-melt, spray-drying, freeze-drying, and the like. Particularly, solid formulations such as tablets, lozenges, and the like can be prepared utilizing such techniques, including wet granulation, co-melt, spray-drying, freeze-drying, and the like.

As set forth previously, the liquid transmucosal delivery systems that can be used with the present invention can take various forms, including, but not limited to, aqueous solutions, non-aqueous solutions, and combinations thereof. Aqueous solutions can include, for example, aqueous gels, aqueous suspensions, aqueous liposomal dispersions, aqueous emulsions, aqueous microemulsions, and combinations thereof. Non-aqueous solutions can include, for example, non-aqueous gels, non-aqueous suspensions, non-aqueous liposomal dispersions, non-aqueous emulsions, non-aqueous microemulsions, and combinations thereof.

In the present invention, the pH of the compositions can be maintained from about pH 2.0 to about pH 10.0 (e.g., 2.0, 3.0, 4.0, 5.0, 6.0, 7.0, 8.0, 9.0, or 10 pH). Compositions having a pH of less than about pH 2.0 (e.g., 0.01, 0.1, 0.25, 0.75, 1, 1.25, 1.50, or 1.75 pH) or greater than about pH 10.0 (e.g., 10.25, 10.50, 10.75, 11, 11.25, 11.5, 12, or 12.5 pH) can increase the risk of irritating the mucosal membranes in the nasal, sublingual, and buccal region of a recipient. Further, the pH of the compositions can be maintained from about pH 3.0 to about pH 7.0 (e.g., 3.0, 3.5, 4.0, 4.5, 5.0, 5.5, 6.0, 6.5, or 7.0 pH). To extend shelf life, preservatives can be added to the present compositions. Suitable preservatives that can be used with the present compositions can include, without limitation, benzyl alcohol, parabens, thimerosal, chlorobutanol, and benzalkonium chloride. The preservative can be present in a composition in a concentration of up to about 0% and about 2%. For example, the preservative can be present in about 0.01%, 0.25%, 0.5%, 0.75%, 1.0%, 1.25%, 1.5%, 1.75% or 2.0%. by weight. The exact concentration of the preservative, however, will vary depending upon the intended use and can be easily ascertained by one skilled in the art.

The present invention provides for the compositions as described above, which are administered through sublingual, buccal, and/or oromucosal membranes to a human or animal to treat ALS or a related condition and/or symptoms of ALS or a related condition.

This invention can also include pharmaceutical compositions, which can contain active agents and one or more pharmaceutically acceptable carriers or excipients. The excipient employed can be one suitable for administration to human or animal subjects. In making the compositions of this invention, the active ingredient can be mixed with an excipient and/or diluted by an excipient. When the excipient serves as a diluent, it can be a solid, semi-solid, or liquid material, which can act as a vehicle, carrier, or medium for the active ingredient.

The compositions of the invention can be formulated to provide fast and/or sustained release of the active ingredient after administration to the patient by employing procedures known in the art.

### Active Agents

The present invention comprises compositions for treating ALS, which can include two or more active agents or compounds derived from Cannabis and methods for using these compositions. Each active compound can make up between about 0.0001% to about 99% by weight of the composition mixture. For example, the active compound(s) can be present in about 0.0001%, 1.0%, 5.0%, 10%, 20%, 30%, 40%, 50%, 60%, 70%, 80%, 90%, or 99% by weight of the composition mixture. The Cannabis compounds, when combined, can have a synergistic effect. In some embodiments, the composition can include at least three, four, five, or more active compounds, in any effective combination. The compositions of the present invention can include any of the following Cannabis compounds listed below, or mixtures thereof:

TABLE 1
Exemplary Cannabinoids

| | |
|---|---|
| THC (tetrahydrocannabinol) | THCA (tetrahydrocannabinolic acid) |
| CBD (cannabidiol) | CBDA (cannabidiolic acid) |
| CBN (cannabinol) | CBG (cannabigerol) |
| CBC (cannabichromene) | CBL (cannabicyclol) |
| CBV (cannabivarin) | THCV (tetrahydrocannabivarin) |
| CBDV (cannabidivarin) | CBCV (cannabichromevarin) |
| CBGV (cannabigerovarin) | CBGM (cannabigerol monomethyl ether) |
| CBE (cannabielsoin) | CBT (cannabicitran) |
| CBCA (cannabichromenic | CBGA (cannabigerolic acid) |
| acid) | |

TABLE 2
Exemplary Terpenes

| | | | |
|---|---|---|---|
| 3-Carene | Alpha Pinene | Beta | Beta Pinene |
| | | Caryophyllene | |
| Bisabolol | Borneol | Camphene | Camphor |
| Caryophyllene | Caryophyllene Oxide | Cedrene | Cedrol |
| Delta 3 Carene | Eucalyptol | Fenchol | Fenchone |
| Geraniol | Geranyl Acetate | Guaiol | Humulene |
| Isoborneol | Isopulegol | Limonene | Linalool |
| Menthol | Myrcene | Nerol | Nerolidol |
| Ocimene | Phellandrene | Phytol | Guaiol |
| Sabinene | Terpinene | Terpineol | Terpinolene |
| Valencene | Caryophyllene | Beta-eudesmol | Eugenol |
| | oxide | | |

TABLE 3
Exemplary Flavonoids

| | | | |
|---|---|---|---|
| Cannflavin A | Cannflavin B | Cannflavin C | Orientin |
| Quercetin | Silymarin | Kaempferol | |

“Compound derived from a Cannabis plant,” as used herein, can be defined as a compound naturally found in Cannabis. The actual compound used in the composition that is naturally found in Cannabis can be produced from another source.

In some embodiments, the composition can include THC and/or CBD. In some embodiments, the composition can include beta-caryophyllene, linalool, and/or alpha-pinene. In some embodiments, the composition can include cannflavin A.

In some embodiments, the composition or formulation including the composition can include about 1%, 5%, 10%, 15%, 20%, 25%, 30%, 35%, 40%, 45%, 50%, 55%, 60%, 65%, 70%, 75%, 80%, 85%, 90%, or more cannabinoid by weight.

In some embodiments, the composition or formulation including the composition can include about 0.001%, 0.01%, 0.1%, 1%, 5%, 10%, 15%, 20%, 25%, 30%, 35%, 40%, 45%, 50%, or more terpenoid or terpene by weight.

In some embodiments, the composition or formulation including the composition can include about 0.0001%, 0.001%, 0.01%, 0.1%, 1%, 5%, or more flavonoid by weight.

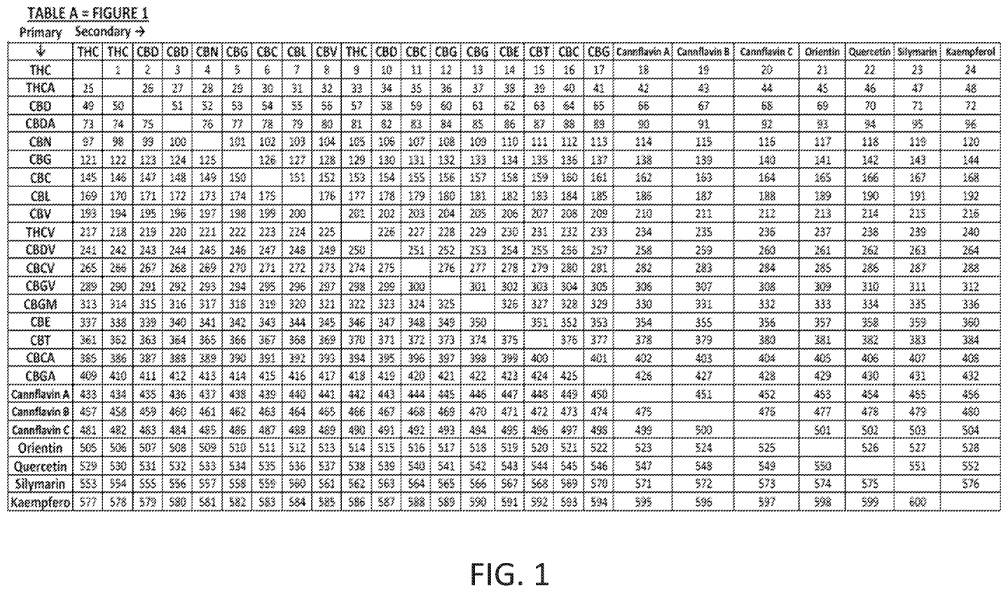

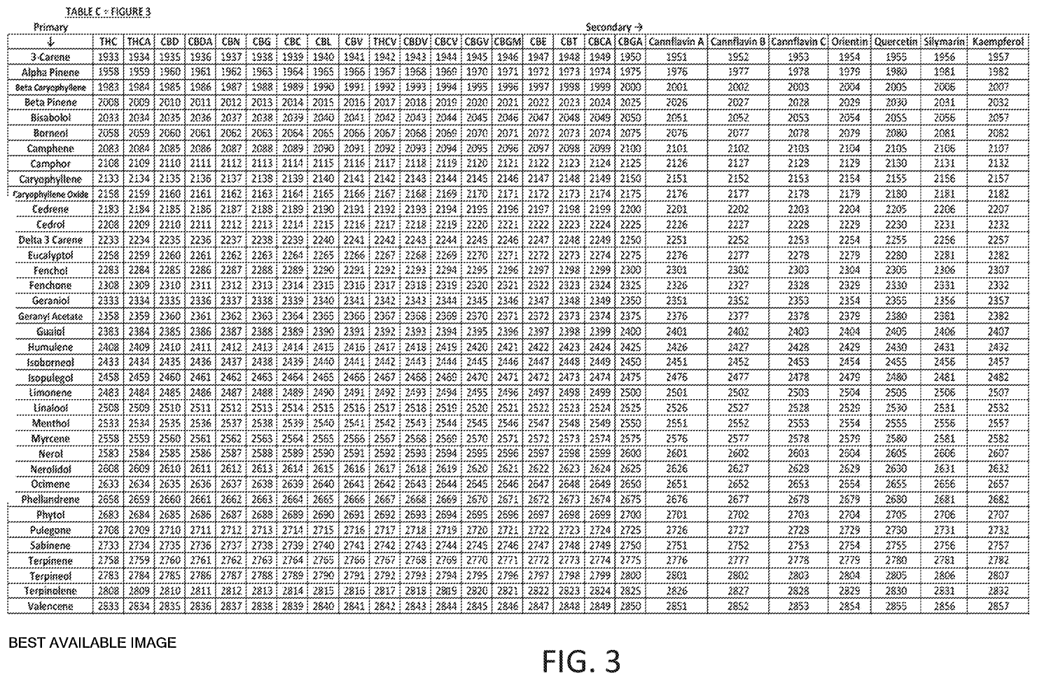

The two compounds together can have synergistic effects of treatment of ALS as provided in the Figures. For example, Formula 1 in FIG. 1 includes a combination of THC and THCA which exhibits synergistic effects. For example, Formula 1933 in FIG. 3 includes a combination of 3-Carene and THC which exhibits synergistic effects.

The use of the terms, “synergistic” and/or “synergistically effective” are used in the present invention to mean a biological effect created from the application of two or more agents to produce a biological effect that is greater than the sum of the biological effects produced by the application of the individual agents. Quantification of synergistic effects can be found in or adapted from S. R. Colby, “Calculating Synergistic and Antagonistic Response of Herbicide Combinations” Weeds 15(1): 20-23, 1967; the entire contents of the foregoing is fully incorporated by reference herein.

While embodiments of the invention can include active ingredients, carriers, inert ingredients, and other formulation components, preferred embodiments can begin with a primary blend. A primary blend can preferably be a synergistic combination containing two or more active ingredients and, optionally, additional ingredients. The primary blends can then be combined with other ingredients to produce a formulation. Accordingly, where concentrations, concentration ranges, or amounts, are given herein, such quantities are in reference to a primary blend or blends. Thus, when a primary blend is further modified by addition of other ingredients to produce a formulation, the concentrations of the active ingredients are reduced proportional to the presence of other ingredients in the formulation.

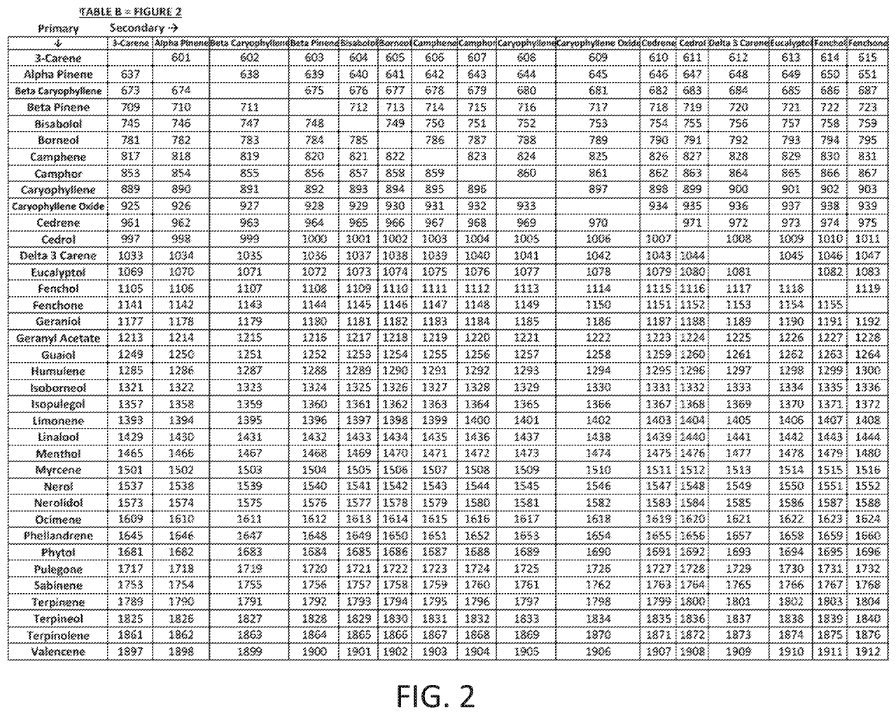

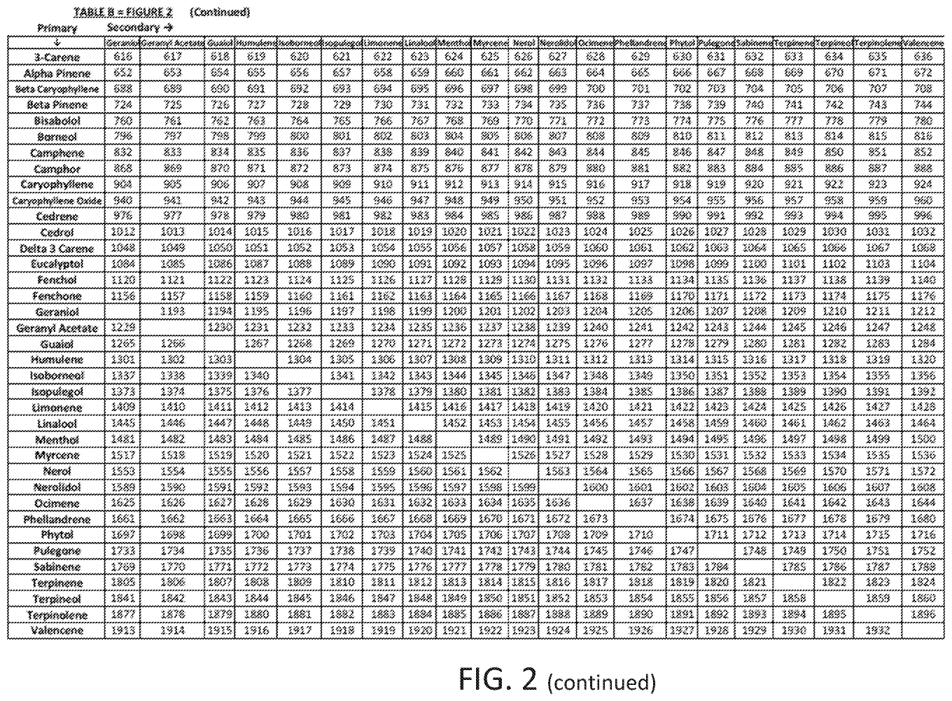

FIGS. 1-3 provide tables of pairwise combinations of synergistic ingredients of the invention. FIG. 1 provides combinations wherein the synergistic ingredients are cannabinoids, flavonoids, or a cannabinoid and a flavonoid. FIG. 2 provides combinations wherein the synergistic ingredients are terpenes. FIG. 3 provides combinations wherein the synergistic ingredients are a terpene and a cannabinoid or a terpene and a flavonoid. For example, in some embodiments, the synergistic ingredients can be THC and CBD (Formula 2). In other embodiments, the synergistic ingredients can be THC and beta-caryophyllene (Formula 1983). In other embodiments, the synergistic ingredients can be CBD and beta-caryophyllene (Formula 1984). In other embodiments, the synergistic ingredients can be CBD and linalool (Formula 2510). In other embodiments, the synergistic ingredients can be THC and alpha-pinene (Formula 1958).

The ratio of the synergistic ingredients can be about 1:1, 1:2, 1:3, 1:6, 1:9, 1:12, 2:1, 2:3, 2:9, 3:1, 3:2, 3:12, 6:1, 9:1, 9:2, 9:12, 12:1, 12:3, 12:9, or more or less.

For example, the formulation can exist in a range of concentration ratios such as 3:1, 1:1, or 1:3. Likewise, the optimized formula (as described in Examples 2-4) can be 12 (or more):1, 9:1, 3:1, 2:1, 1.5:1, 1:1, 1:1.5, 1:2, 1:3, 1:6, 1:9, 1:12 (or more), or the like.

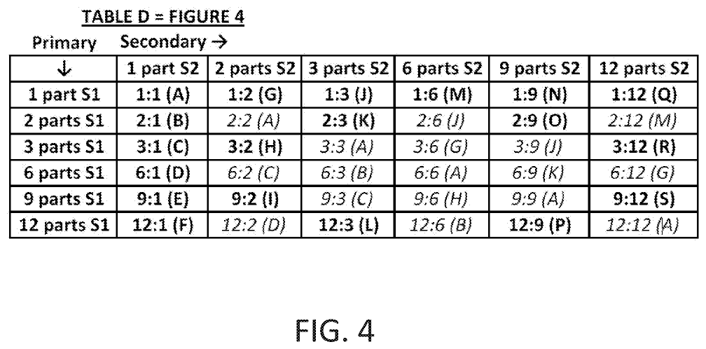

FIG. 4 lists major ratios of pairwise combinations of synergistic ingredients of the invention, named as ratios A through S. While the table in this figure provides a range of ratios that can be useful, it is within the scope of the invention to provide formulations in which synergistic ratios are adapted to particular uses.

In other embodiments, the ratio of the synergistic ingredients can range from 10:1 to 1:5000 or less. For example, the ratio can be about 10:1, 1:1, 1:10, 1:500, 1:1000, 1:1500, 1:2000, 1:2500, 1:3000, 1:3500, 1:4000, 1:4500, or 1:5000, depending on the synergistic ingredients.

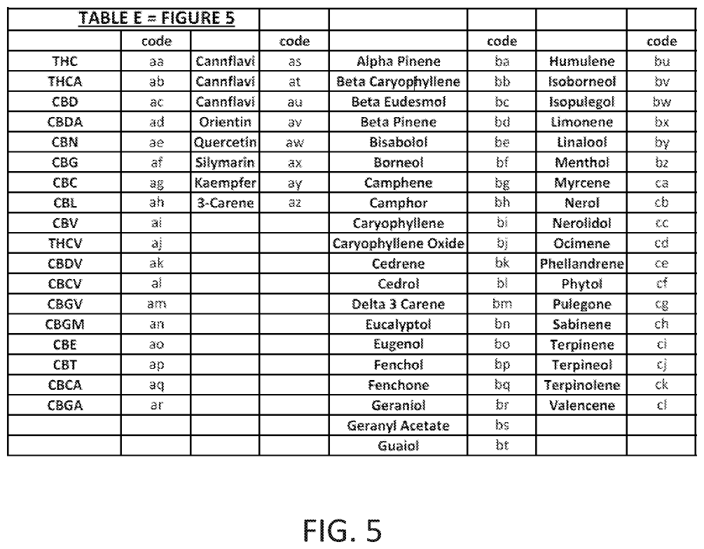

Likewise, the synergistic formulations of the invention can include additional ingredients to further synergistically enhance the efficacy of a pairwise combination. In such embodiments, a paired combination can be enhanced with a tertiary ingredient selected from FIG. 5. In some embodiments, a quaternary ingredient can also be selected from FIG. 5. Likewise, in other embodiments, additional synergists can be selected, either from FIG. 5 or from other sources based upon desired effects.

In some embodiments, the active agents can include a cannabinoid or derivative thereof. The cannabinoid can be, for example, one or more of cannabigerolic acid (CBGA), cannabigerolic acid monomethylether (CBGAM), cannabigerol (CBG), cannabigerol monomethylether (CBGM), cannabigerovarinic acid (CBGVA), cannabigerovarin (CBGV), cannabichromenic acid (CBCA), cannabichromene (CBC), cannabichromevarinic acid (CBCVA), cannabichromevarin (CBCV), cannabidiolic acid (CBDA), cannabidiol (CBD), cannabidiol monomethylether (CBDM), cannabidiol-C4 (CBD-C4), cannabidivarinic acid (CBDVA), cannabidivarin (CBDV), cannabidiorcol (CBD-C1), delta-9-tetrahydrocannabinolic acid A (THCA-A), delta-9-tetrahydrocannabinolic acid B (THCA-B), delta-9-tetrahydrocannabinol (THC), delta-9-tetrahydrocannabinolic acid-C4 (THCA-C4), delta-9-tetrahydrocannabinol-C4 (THC-C4), delta9-tetrahydrocannabivarinic acid (THCVA), delta-9-tetrahydrocannabivarin (THCV), delta-9-tetrahydrocannabiorcolic acid (THCA-C1), delta-9-tetrahydrocannabiorcol (THC-C1), delta-7-cis-iso-tetrahydrocannabivarin, delta-8-tetrahydrocannabinolic acid (A8-THCA), delta-8-tetrahydrocannabinol (A8-THC), cannabicyclolic acid (CBLA), cannabicyclol (CBL), cannabicyclovarin (CBLV), cannnabielsoic acid A (CBEA-A), cannabielsoic acid B (CBEA-B), cannabielsoin (CBE), cannabinolic acid (CBNA), cannabinol (CBN), cannabinol methylether (CBNM), cannabinol-C4 (CBN-C4), cannabivarin (CBV), cannabinol-C2 (CBN C2), cannabiorcol (CBN-C1), cannabinodiol (CBND), cannabinodivarin (CBVD), cannabitriol (CBT), 10-ethoxy-9-hydroxy-delta-6a-tetrahydrocannabinol, 8,9-dihydroxy-delta-6atetrahydrocannabinol, cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-delta-6a-tetrahydrocannabinol (OTHC), delta-9-cis-tetrahydrocannabinol (cisTHC), 3,4,5,6-tetrahydro-7-hydroxy-alpha-alpha-2-trimethyl-9-n-propyl-2,6-methano-2H-1-benzoxocin-5-methanol (OH-iso-HHCV), cannabiripsol (CBR) and trihydroxy-delta-9-tetrahydrocannabinol (triOH-THC), or the like.

In some embodiments, the active agent can include a terpene or derivative thereof. The terpene can be myrcene, limonene, linalool, ocimene, beta-pinene, alpha-pinene, betacaryophyllene, humulene, delta-3-carene, gamma-bisabolene, alpha-farnesene, beta-fenchol, guaiol, alpha-guaiene, terpinolene, beta-eudesmol, alphabergamotene, epi-alpha-bisabolol and caryophyllene oxide, and/or the like.

In some embodiments, the composition can include one or more oil(s). The oil can be a liquid hydrocarbon-based oil. Non-limiting examples of oils include plant-based oils, mineral oils, triglycerides, essential oils, and animal-based oils. In some embodiments, the oil can include, but is not limited to, plant-based oil, mineral oil, triglyceride, essential oil, animal-based oil, and/or the like. In other embodiments, the oil can include, but need not be limited to, light mineral oil, medium-chain triglyceride (MCT) oil, hydrogenated castor oil, jojoba oil, peppermint oil, Cannabis oil, and/or the like.

In some embodiments, the composition can include a viscosity-controlling agent/emulsifier. Suitable emulsifiers can be, for example, hydrogenated lecithin, glycerin, sodium gluconate, acrylates, C10-30 alkyl acrylate crosspolymer, sodium carboxymethyl betaglucan, castor oil, polyglyceryl-3 methylglucose distearate, cetearyl alcohol, behenyl alcohol, butylene glycol, propylene glycol, xanthan gum, potassium cetyl phosphate, polyglyceryl-6 distearate jojoba esters, polyglyceryl-3 beeswax, cetyl alcohol, PEG-800, laureth-7, C13-14 isoparrafin, polyisobutene, sodium carboxymethyl betaglucan, PEG-200 hydrogenated glyceryl palmate, cellulose gum, PEG-7 glyceryl cocoate, aluminum starch octenylsuccinate, and/or the like.

In some embodiments, the composition can include a solvent. These solvents can include, for example, acetone dichloromethane, acetonitrile, n-butyl ether, monomethylacetamide, dipropylene glycol monomethyl ether, diethyl phthalate fatty acid esters such as the diethyl ester or diisobutyl adipate, water, alkanol, benzyl benzoate, dipropylene glycol monomethyl ether, diethylene glycol monobutyl ether, silicone, dimethylacetamide, 2,2-dimethyl-4-oxy-methylenel,3-dioxolane. N,N-dimethylalkanamides (e.g., N,N dimethylformamide), limonene, eucalyptol, dimethyl sulfoxide, alkylpyrrolidones (e.g., N-methylpyrrolidone, 2-pyrrolidone), liquid polyoxyethylene glycols, methylene glycol, ethylene glycol, propylene glycol, dipropylene glycol, polypropylene glycol, butyl diglycol, dipropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, triacetin, aromatic hydrocarbons, dearomatized aliphatics, alkylbenzenes, alkylnaphthalenes, ketones such as methyl ethyl ketone, cyclohexanone, 2-heptanone, isophorone and 4-hydroxy-4-methyl-2-pentanone, acetates such as ethyl acetate, benzyl acetate, isoamyl acetate, hexyl acetate, heptyl acetate, octyl acetate, nonyl acetate, tridecyl acetate and isobornyl acetate, other esters such as alkylated lactate esters, dibasic esters and 7-butyrolactone, and alcohols, which can be linear, branched, saturated or unsaturated, such as phenyl ethyl alcohol, methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol and benzyl alcohol. Such solvents also include glycerol esters of saturated and unsaturated fatty acids (e.g., C₆-C₂₂), such as plant seed and fruit e.g., oils of olive, castor, linseed, sesame, corn (maize), peanut, sunflower, grapeseed, safflower, cottonseed, soybean, rapeseed, coconut and palm kernel, and mixtures thereof, e.g., polyethoxylated castor oil. Such solvents can also include alkylated fatty acids (e.g., methylated, ethylated, butylated) wherein the fatty acids can be obtained by hydrolysis of glycerol esters from plant and animal sources, and can be purified by distillation.

In some embodiments, the composition can include THC, CBD, CBG, THCA, CBDA, beta-caryophyllene, alpha-pinene, d-limonene, linalool, and/or the like.

In some embodiments, the composition can provide a synergistic effect with respect to treatment/inhibition of ALS as compared to the effect provided by the components of the composition administered separately in similar concentrations.

Non-limiting examples of synergistic combinations and their main effects can include:
1) THC plus CBD: antioxidant neuroprotection and to allay disease progression
2) THC plus beta-caryophyllene: analgesia
3) CBD plus beta-caryophyllene: anti-inflammatory
4) CBD plus linalool: anti-anxiety
5) THC plus alpha-pinene: mental clarity/decrease short-term memory impairment by THC
6) Cannabinoid and Cannflavin A: anti-inflammatory

In some embodiments, the composition can treat, inhibit, and/or ameliorate ALS, related diseases, or symptoms of the same.

### Methods of Treatment

Some embodiments of the invention can relate to methods of treating or preventing ALS, including but not limited to, administering the composition or formulation to a subject in a therapeutically effective dosage, wherein the administration of the composition achieves any of the effects described in this disclosure. In some embodiments, the subject can experience at least one of prevention of ALS or a related condition, reduction of any ALS disease symptoms, and/or an accelerated recovery from ALS or a related condition.

In some embodiments, the ALS disease symptoms can include pain, sleep, fatigue, drooling, difficulty chewing or swallowing, difficulty speaking, spasticity, muscle twitching, muscle spasms, muscle cramps, constipation, weight loss, shortness of breath, and/or the like.

In some embodiments, the composition can be administered once, twice, three, four, or more times throughout a day. In some embodiments, the composition can be administered over a time period of about 1 day to about 6 months or more (e.g., 1 day, 15 days, 1 month, 2 months, 3 months, 4 months, 6 months, or more).

### REFERENCES

The following references relate to the invention and are hereby incorporated by reference in their entireties: AMTMANN, D., WEYDT, P., JOHNSON, K. L., JENSEN, M. P. & CARTER, G. T. 2004. Survey of cannabis use in patients with amyotrophic lateral sclerosis. Am J Hosp Palliat Care, 21, 95-104; BENEDUSI, V., MARTORANA, F., BRAMBILLA, L., MAGGI, A. & ROSSI, D. 2012. The peroxisome proliferator-activated receptor gamma (PPARgamma) controls natural protective mechanisms against lipid peroxidation in amyotrophic lateral sclerosis. J Biol Chem, 287, 35899-911; BRIERLEY, D. I., HARMAN, J. R., GIALLOUROU, N., LEISHMAN, E., ROASHAN, A. E., MELLOWS, B. A. D., BRADSHAW, H. B., SWANN, J. R., PATEL, K., WHALLEY, B. J. & WILLIAMS, C. M. 2019. Chemotherapy-induced cachexia dysregulates hypothalamic and systemic lipoamines and is attenuated by cannabigerol. J Cachexia Sarcopenia Muscle, 10, 844-859′ BRIERLEY, D. I., SAMUELS, J., DUNCAN, M., WHALLEY, B. J. & WILLIAMS, C. M. 2017. A cannabigerol-rich Cannabis sativa extract, devoid of [INCREMENT]9-tetrahydrocannabinol, elicits hyperphagia in rats. Behav Pharmacol, 28, 280-284, BROOKS, B. R., MILLER, R. G., SWASH, M., MUNSAT, T. L. & WORLD FEDERATION OF NEUROLOGY RESEARCH GROUP ON MOTOR NEURON, D. 2000. E1 Escorial revisited: 5 revised criteria for the diagnosis of amyotrophic lateral sclerosis. Amyotroph Lateral Scler Other Motor Neuron Disord, 1, 293-9; CARTER, G. T., ABOOD, M. E., AGGARWAL, S. K. & WEISS, M. D. 2010. Cannabis and amyotrophic lateral sclerosis: hypothetical and practical applications, and a call for clinical trials. Am J Hosp Palliat Care, 27, 347-56; CARTER, G. T. & ROSEN, B. S. 2001. Marijuana in the management of amyotrophic lateral sclerosis. Am J Hosp Palliat Care, 18, 264-70; GUGLIANDOLO, A., POLLASTRO, F., GRASSI, G., BRAMANTI, P. & MAZZON, E. 2018. In Vitro Model of Neuroinflammation: Efficacy of Cannabigerol, a NonPsychoactive Cannabinoid. Int J Mol Sci, 19; HAIDER, A., SPINELLI, F., HERDE, A. M., MU, B., KELLER, C., MARGELISCH, M., WEBER, M., SCHIBLI, R., MU, L. & AMETAMEY, S. M. 2018. Evaluation of 4-oxo-quinoline-based CB2 PET radioligands in R6/2 chorea huntington mouse model and human ALS spinal cord tissue. Eur J Med Chem, 145, 746-759; JOARDAR, A., MENZL, J., PODOLSKY, T. C., MANZO, E., ESTES, P. S., ASHFORD, S. & ZARNESCU, D. C. 2015. PPAR gamma activation is neuroprotective in a Drosophila model of ALS based on TDP43. Hum Mol Genet, 24, 1741-54; LECARPENTIER, Y. & VALLEE, A. 2016. Opposite Interplay between PPAR Gamma and Canonical Wnt/Beta-Catenin Pathway in Amyotrophic Lateral Sclerosis. Front Neurol, 7, 100; LEWIS, M. A., RUSSO, E. B. & SMITH, K. M. 2018. Pharmacological Foundations of Cannabis Chemovars. Planta Med, 84, 225-233; MAMMANA, S., CAVALLI, E., GUGLIANDOLO, A., SILVESTRO, S., POLLASTRO, F., BRAMANTI, P. & MAZZON, E. 2019. Could the Combination of Two Non-Psychotropic Cannabinoids Counteract Neuroinflammation?Effectiveness of Cannabidiol Associated with Cannabigerol. Medicina (Kaunas), 55; MEYER, T., FUNKE, A., MUNCH, C., KETTEMANN, D., MAIER, A., WALTER, B., THOMAS, A. & SPITTEL, S. 2019. Real world experience of patients with amyotrophic lateral sclerosis (ALS) in the treatment of spasticity using tetrahydrocannabinol:cannabidiol (THC:CBD). BMC Neurol, 19, 222; NAHLER, G. 2017. Comedication with cannabidiol may slow down the progression of motor neuron disease: A case report. J Gen Pract, 5, 1-3; NG, L., KHAN, F., YOUNG, C. A. & GALEA, M. 2017. Symptomatic treatments for amyotrophic lateral sclerosis/motor neuron disease. Cochrane Database Syst Rev, 1, CD011776; NICHOLSON, K., MURPHY, A., MCDONNELL, E., SHAPIRO, J., SIMPSON, E., GLASS, J., MITSUMOTO, H., FORSHEW, D., MILLER, R. & ATASSI, N. 2018. Improving symptom management for people with amyotrophic lateral sclerosis. Muscle Nerve, 57, 20-24; RIVA, N., MORA, G., SORARU, G., LUNETTA, C., FERRARO, O. E., FALZONE, Y., 6 LEOCANI, L., FAZIO, R., COMOLA, M., COMI, G. & GROUP, C. S. 2019. Safety and efficacy of nabiximols on spasticity symptoms in patients with motor neuron disease (CANALS): a multicentre, double-blind, randomised, placebo-controlled, phase 2 trial. Lancet Neurol, 18, 155-164; RODRIGUEZ-CUETO, C., SANTOS-GARCIA, I., GARCIA-TOSCANO, L., ESPEJOPORRAS, F., BELLIDO, M., FERNANDEZ-RUIZ, J., MUNOZ, E. & DE LAGO, E. 2018. Neuroprotective effects of the cannabigerol quinone derivative VCE-003.2 in SOD1(G93A) transgenic mice, an experimental model of amyotrophic lateral sclerosis. Biochem Pharmacol, 157, 217-226; RUSSO, E. B. 2011. Taming THC: potential cannabis synergy and phytocannabinoid-terpenoid entourage effects. Br J Pharmacol, 163, 1344-64; RUSSO, E. B. 2018. Cannabis Therapeutics and the Future of Neurology. Frontiers in Integrative Neuroscience, 12, 1-11; SHOEMAKER, J. L., SEELY, K. A., REED, R. L., CROW, J. P. & PRATHER, P. L. 2007. The CB2 cannabinoid agonist AM-1241 prolongs survival in a transgenic mouse model of amyotrophic lateral sclerosis when initiated at symptom onset. J Neurochem, 101, 87-98; URBI, B., BROADLEY, S., BEDLACK, R., RUSSO, E. & SABET, A. 2019. Study protocol for a randomised, double-blind, placebo-controlled study evaluating the Efficacy of cannabis-based Medicine Extract in slowing the disease progression of Amyotrophic Lateral sclerosis or motor neurone Disease: the EMERALD trial. BMJ Open, 9, e029449; VAN DAMME, P., ROBBERECHT, W. & VAN DEN BOSCH, L. 2017. Modelling amyotrophic lateral sclerosis: progress and possibilities. Dis Model Mech, 10, 537-549; WEBER, M., GOLDMAN, B. & TRUNIGER, S. 2010. Tetrahydrocannabinol (THC) for cramps in amyotrophic lateral sclerosis: a randomised, double-blind crossover trial. J Neurol Neurosurg Psychiatry, 81, 1135-40.

### EXAMPLES

The following examples are offered to illustrate this invention and are not to be construed in any way as limiting the scope of this invention.

### Example 1

### Demonstration of Synergy

Experiments are done to demonstrate synergy of the components of the composition. Quantification of putative synergistic effects is based upon an adaptation of the Colby method (S. R. Colby, “Calculating Synergistic and Antagonistic Response of Herbicide Combinations” Weeds, 15(1): 20-23, 1967). Specifically, each putative synergistic agent is administered to cells in an in vitro model and/or to animals in an animal model of the disease process, and the quantitative response is recorded. Then the combination of the active agents is made at 3:1, 1:1, and 1:3 ratios and each combination is contacted with the model assay system along a dilution gradient. The response of each treatment is quantified and a calculation of synergy is derived. Synergistic combinations in terms of ratios of the two components and in terms of dose-response are further optimized in subsequent tests based upon the first series of tests. Accordingly, a synergistic formulation and dosing protocol is developed.

### Example 2

### Formulation

In this example, the composition includes 2.5 mg THC, 75 mg CBD, 10 mg CBG, 2.5 mg THCA, 2.5 mg CBDA, 10 mg beta-caryophyllene, 2 mg alpha-pinene, 5 mg D-limonene, and 3 mg linalool for a total of 112.5 mg cannabinoids and terpenoids combined with qs organic MCT or coconut oil=1 ml of oromucosal medicine.

### Example 3

### Method

A formulation described herein is delivered by topical spray to a patient. The topical spray contacts the oral mucosa and throat. In addition, or as an alternative, the formulation is delivered by inhaled mist or other respiratory delivery system to the bronchus and lungs. The patient experiences at least one of prevention of ALS or a related condition, reduction of any ALS disease symptoms, and/or an accelerated recovery from ALS or a related condition.

### Example 4

A transmucosal formulation including THC and CBD is prepared and found to have synergistic effects. The formulation is found to provide antioxidant neuroprotection and to allay disease progression.

### Example 5

A transmucosal formulation including THC and beta-caryophyllene is prepared and found to have synergistic effects. The formulation is found to provide analgesia.

### Example 6

A transmucosal formulation including CBD and beta-caryophyllene is prepared and found to have synergistic effects. The formula is found to have anti-inflammatory effects.

### Example 7

A transmucosal formulation including CBD and linalool is prepared and found to have synergistic effects. The formulation is found to have anti-anxiety effects.

### Example 8

A transmucosal formulation including CBD and alpha-pinene is prepared and found to have synergistic effects. The formulation is found to provide mental clarity/decreased short-term memory impairment by THC.

The various methods and techniques described above provide a number of ways to carry out the application. Of course, it is to be understood that not necessarily all objectives or advantages described are achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by including one, another, or several other features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, any numbers expressing quantities of ingredients, properties such as molecular weight, reaction conditions, and so forth, used to describe and claim certain embodiments of the disclosure are to be understood as being modified in some instances by the term “about.” Accordingly, in some embodiments, the numerical parameters set forth in the written description and any included claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are usually reported as precisely as practicable.

In some embodiments, the terms “a” and “an” and “the” and similar references used in the context of describing a particular embodiment of the application (especially in the context of certain claims) are construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Variations on preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

What is claimed is:

1. A composition comprising a combination of at least two active agents extracted from Cannabis and a pharmaceutically acceptable carrier, wherein the composition is provided in a form suitable for transmucosal administration, wherein the two active agents have a synergistic effect on the treatment of ALS or a related condition and/or symptoms thereof.

2. The composition of claim 1, wherein at least one of the active agents is a cannabinoid, a terpene, or a flavonoid.

3. The composition of claim 1, wherein the active agents are selected from the group consisting of THC, CBD, CBG, THCA, CBDA, beta-caryophyllene, alpha-pinene, D-limonene, and linalool.

4. The composition of claim 1, comprising THC, CBD, CBG, THCA, CBDA, beta-caryophyllene, alpha-pinene, D-limonene, and linalool.

5. The composition of claim 1, wherein the active agents comprise THC and CBD.

6. The composition of claim 1, wherein the active agents comprise THC and beta-caryophyllene.

7. The composition of claim 1, wherein the active agents comprise CBD and beta-caryophyllene.

8. The composition of claim 1, wherein the active agents comprise CBD and linalool.

9. The composition of claim 1, wherein the active agents comprise THC and alpha-pinene.

10. The composition of claim 1 further comprising cannflavin A.

11. The composition of claim 1 wherein the pharmaceutically acceptable carrier is selected from the group consisting of aqueous solution, non-aqueous solution, or a combination of an aqueous solution and a non-aqueous solution thereof.

12. The composition of claim 11 wherein the pharmaceutically acceptable carrier comprises a solution, gel, suspension, liposomal dispersion, emulsion, microemulsion, nanoparticle, or combinations thereof.

13. The composition of claim 1 wherein the composition is provided in a sublingual or buccal or oromucosal transmucosal liquid dosage form.

14. The composition of claim 13 wherein the transmucosal liquid dosage form is selected from the group consisting of: a spray and liquid drops.

15. The composition of claim 1 wherein the composition is provided in a sublingual or buccal or oromucosal transmucosal solid dosage form.

16. The composition of claim 15 wherein the transmucosal solid dosage form is selected from the group consisting of a tablet, a chewing gum, a patch, a lozenge, a troche, a pastille, a sachet, and a rapid disintegrating tablet or strip.

17. The composition of claim 1 wherein the composition further comprises at least one of: flavor agent, artificial coloring, sweetener, lubricating agent, disintegration agent, permeation enhancer, lubricating agent, diluent, base, or buffering agent.

18. The composition of claim 1, further comprising an additional active agent.

19. A method for the treatment of ALS or a related condition and/or symptoms of ALS or a related condition, comprising transmucosally administrating a therapeutically effective amount of the composition of claim 1 to a subject in need of such treatment, wherein ALS or a related condition and/or symptoms of ALS or a related condition are ameliorated.

20. The method of claim 19 wherein the pharmaceutical composition is administered transmucosally through sublingual, buccal, or oromucosal routes of delivery.
